(19) United States
(12) Plant Patent Application Publication
(10) Pub. No.: US 2016/0044845 P1
(43) Pub. Date: Feb. 11, 2016

(54) Grapevine 'Sheegene-24'

(50) ;

(71) Applicant: Sheehan Genetics, LLC, Fresno, CA (US)

(72) Inventor: Timothy P. Sheehan, (US)

(21) Appl. No.: 14/121,181

(22) Filed: Aug. 8, 2014

(51) Int. Cl. A01H 5/00

(52) U.S. Cl. PLT/207

(57) ABSTRACT

A new and distinct variety of grapevine characterized by the production of large, green-colored seeded grapes that mature for harvesting and shipment in mid-September to mid-October when grown in the San Joaquin Valley of Central California. The grapes of this new variety have a very crisp texture and excellent eating quality.

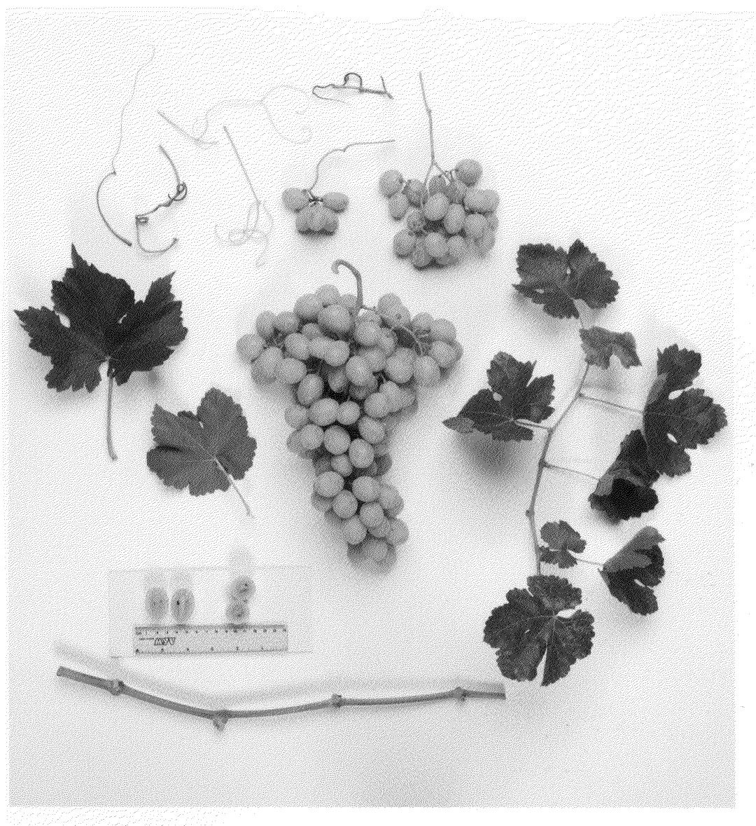

### CROSS REFERENCE TO RELATED APPLICATIONS

The claimed plant is not subject of a related application.

### STATEMENT OF ANY FEDERALLY-SPONSORED RESEARCH AND DEVELOPMENT

The claimed plant is not subject of Federally-sponsored research or development.

### LATIN NAME OF THE GENIUS AND SPECIES OF THE PLANT CLAIMED

The claimed plant relates to a new and distinct variety of Vitis vinifera to be known as ‘Sheegene-24’.

### VARIETY DENOMINATION

The new variety of Vitis vinifera is the result of hybridization of ‘Princess’ (unpatented), the pollen parent, and ‘Red Globe’ (U.S. Plant Pat. No. 4,787), the seed parent. The new variety was first hybridized by Timothy P. Sheehan in the Spring of 2000. The new variety was asexually propagated in the dormant season of 2003, grafted on virus-free rootstock in a Vitis vinifera variety block located East of Fowler, Calif. The new variety was planted as rooted cuttings in the dormant season of 2007 in a Vitis vinifera variety block located north and west of Delano, Calif. The new variety produces a large, green-colored seeded grape with a very crisp texture and excellent eating quality. The new variety has been shown to maintain its distinguishing characteristics through asexual propagation.

The new variety is distinguished from its pollen parent, ‘Princess’ (unpatented), in that the new variety produces green-colored seeded grapes, as compared to the white-colored seedless grapes produced by its pollen parent, ‘Princess’ (unpatented). The new variety is distinguished from its seed parent, ‘Red Globe’ (U.S. Plant Pat. No. 4,787), in that the new variety produces large sized green-colored seeded grapes, as compared to its seed parent, ‘Red Globe’ (U.S. Plant Pat. No. 4,787), that produces exceptionally large red-colored seeded grapes.

### BACKGROUND OF THE INVENTION

The present invention relates to a new and distinct variety of grapevine that produces a large, green-colored seeded grape that matures for harvesting and shipment in mid-September to mid-October when grown in the San Joaquin Valley of Central California.

### SUMMARY OF THE INVENTION

The ‘Sheegene-24’ grapevine is characterized by producing a large, green-colored seeded grape with a very crisp texture and it is productive and mature for harvesting and shipment in mid-September to mid-October when grown in the San Joaquin Valley of Central California. The new variety can be compared to ‘Calmeria’ (unpatented) but the grapes produced by the new variety are larger and have a broad ellipsoid shape, as opposed to the elongated narrow ellipsoidal shape of ‘Calmeria’ (unpatented), among other distinguishing characteristics of the new variety.

### BRIEF DESCRIPTION OF THE DRAWINGS

The accompanying drawing is a color photograph of the new variety of grapevine displaying leaves, tendrils, mature primary cluster, tendril cluster, secondary cluster, two grapes cut in half, and a mature shoot(nodes and internodes). Across the top portion of the photograph are tendrils, tendril cluster, and a secondary cluster. Across the middle portion of the photograph are leaves displaying the upper and lower surfaces, a mature primary cluster, and an immature shoot tip with leaves. Across the bottom of the photograph are two grapes cut longitudinally and horizontally displaying the heart and pulp, with a mature shoot (nodes and internodes) shown just below the two cut grapes.

### DETAILED BOTANICAL DESCRIPTION

Referring more to the horticultural description of the new and distinct variety of grapevine, the following has been observed under the ecological conditions prevailing at the origin vineyard located near Delano, Calif., in the San Joaquin Valley of Central California. All major color descriptions are by reference to Munsell Color Charts for Plant Tissues, Munsell Color, New Windsor, N.Y., 1977.
Plant:
   Plant habit and growth.—Moderate vigor, semi-erect shoot growth, heavy secondary shoot growth.
   Size.—Age at maturity: 3 to 4 years. Height (at maturity): Cordon: 120 cm; Canopy: 210 cm. Width (at maturity): 300 cm.
Trunk:
   Size.—Diameter: 7.3 cm measured 45 cm from floor.
   Surface texture.—Coarse, rough.
   Color.—Exposed surface (7.5 YR 5/2); interior surface (2.5 YR 5/6).
Canes:
   Size.—Diameter: 1.2 cm (basilar measurement). Length: 150 cm.
   Surface texture (mature cane).—Smooth.
   Surface texture (immature cane).—Smooth.
   Form (woody shoot cross section form).—Pith in center with diaphragm at nodes.
   Color (mature).—color and indicate. specific age: 5 YR 6/8 at 12 weeks.
   Color (immature).—color and indicate specific age: 5 GY 7/6 at 3 weeks.
   Internode length (upper mature sun cane).—7.5 cm.
Tendrils:
   Form.—Approximately 50 percent (50%) trifid and 50 percent (50%) quadrifid.
   Size.—Small.
   Length.—Average 20 cm.
   Diameter.—Average 2 mm.
   Texture and distribution.—Smooth. (Mid-cane towards tip) 1,1,0,1,1,0,1,1,0.
   Color (mature).—color and indicate specific age: 2.5 GY 8/6 at 5 weeks age.
   Anthocynanin (mature).—Absent.
   Color (immature).—color and indicate specific age: 2.5 GY 8/6 at 2 weeks age.
   Anthocynanin (immature).—Absent.
Growing tips (young shoot):
   Pubescence.—Very slight.
   Color.—2.5 GY 7/6.
   Anthocynanin.—Absent.
   Shape.—Wide open.
   Apex.—Normal, triangular.
Leaves:
   Shape.—Pentagonal; slight cupping upward.
   Apex.—Pointed.
   Base.—Rounded.
   Margin.—Teeth irregular.
   Length of teeth on margin.—1 to 8 mm.
   Shape of teeth on margin.—Both sides convex.
   Texture (mature leaf).—Upper surface: Slightly bullate. Lower surface: Smooth, glabrous.
   Size.—Immature, length: include length and age: 7 cm; 10 days. Immature, width: include width and age: 7 cm; 10 days. Mature, length: include length and age: 11 cm; 20 days. Mature, width: include width and age: 12 cm; 20 days.
   Color, immature leaf.—Upper surface: Shiny; 2.5 GY 8/10. Lower surface: Dull; 2.5 GY 6/8.
   Color, mature leaf.—Upper surface: Shiny; 5 GY 4/8. Lower surface: Dull; 5 GY 5/6.
   Venation.—Pattern: Primary and secondary veins on the upper surface are visible (veins have lighter color than interveinal tissue); veins on lower leaf surface are raised. Color: No anthocyanin pigment. Upper surface: 2.5 GY 8/8. Lower surface: 2.5 GY 8/6.
   Petiolar sinus.—include general description: Mostly half open.
   Petiole.—Length: 7 cm. Diameter: 2 mm. Color: 2.5 GY 6/8.
Floral cluster:
   General description and location.—Floral clusters are large and tapered with an occasional tendril. Primary cluster is mostly on 4th node from base.
   Quantity of inflorescences per cluster.—Abundant (800 to 1,000).
   Size.—Length: From 18 to 24 cm; average 20 cm. Width: From 12 to 20 cm; average 16.5 cm.
   Peduncle.—Length (Average): Length 6 cm; Width 4.5 mm.
   Inflorescences.—Hermaphroditic.
   Stamens (general description).—Straight, 3 mm long.
   Anthers (general description).—Normal size , pale yellow.
   Date of bloom.—Full bloom on April 27; bloom duration approximately 5 days.
   Pollen amount.—Moderate.
   Calyptra.—5 segments; complete separation.
   Calyptra color.—5GY 5/6.
Fruit:
   Time of year of commercial harvest and shipment.—Mid-September to Mid-October.
   Cluster (primary bunches).—Size: Large. Length (without peduncle): From 20 to 29 cm; average 25 cm. Width: From 15 to 26 cm; average 23 cm. Density: Medium. Peduncle length: From 5 to 8 cm; average 7 cm. Peduncle diameter (Average): 5 mm. Peduncle color: (5 GY 6/4). Number of berries per cluster (Average): 125. Berry: Size: Large. Shape: Broad ellipsoid. Uniformity: Good. Brix content: 16.4° on August 15; 20° on September 30. Weight: 8.5 g on August 15; 12.4 g on September 30. Diameter: 23 mm on August 15; 29 mm on September 30. Length: 30 mm on August 15; 38 mm on September 30. Skin color: (2.5 GY 7/6). Pedicel: Length: 11.5 mm. Diameter: 2 mm. Color: 2.5 GY 7/8. Strength of attachment to berry: Strong.
   Cluster (secondary bunches).—Scarce. Size: Small. Length (without peduncle): 6 cm. Width: 6 cm. Density: Medium. Peduncle length: 6.5 cm. Peduncle diameter: 3 mm. Peduncle color: 2.5 GY 7/6. Number of berries per cluster (Average): 22. Berry: Size: Medium. Weight: 2 g. Shape: Broad ellipsoid. Uniformity: Good. Brix content: 5.4° on September 30. Size: Diameter: 15 mm. Length: 20 mm. Skin color: 5 GY 6/6. Pedicel: Length: 10 mm. Diameter: 1.5 mm. Color: 5 GY 6/6. Strength of attachment to berry: Strong.
Berry flesh:
   Color.—2.5 GY 8/2.
   Juice, color.—Clear.
   Juice production.—Poor.
   Thickness of skin.—Medium.
   Flavor.—Neutral.
   Fragrance.—Neutral.
   Texture.—Very Crisp; excellent eating quality.
   Seeds (Average).—2 seeds per berry.
   Seed (color).—5 YR 4/4.
   Seed (Length).—6 mm.
   Seed (Width).—3 mm.
   Use.—Fresh Table.
Disease and insect resistance: No particular resistance or susceptibility has been observed. Normal disease control practices can be used.

Having thus described and illustrated our new variety of grapevine, we claim:

1. A new and distinct variety of grapevine, substantially as described and illustrated herein, characterized principally by its production of large, green-colored seeded grapes.
